(19) United States
(12) Patent Application Publication
(10) Pub. No.: US 2020/0403159 A1
(43) Pub. Date: Dec. 24, 2020

(54) COMPOUND, AND ORGANIC LIGHT-EMITTING ELEMENT, DISPLAY PANEL AND DISPLAY DEVICE INCLUDING THE SAME

(71) Applicant: Shanghai Tianma AM-OLED Co., Ltd., Shanghai (CN)

(72) Inventors: Lei ZHANG, Shanghai (CN); Wei Gao, Shanghai (CN); Jinghua Niu, Shanghai (CN); Wenpeng Dai, Shanghai (CN); Quan Ran, Shanghai (CN); Xia Li, Shanghai (CN)

(21) Appl. No.: 17/011,988

(22) Filed: Sep. 3, 2020

(30) Foreign Application Priority Data
Jun. 10, 2020 (CN) 202010524204.0

(51) Int. Cl. H01L 51/00; C07D 213/16; C09K 11/06; C07D 251/24; C07D 405/10; C07D 401/10; C07D 405/04; C07D 403/10; C07D 401/04; C07D 401/14; C07D 405/14

(52) U.S. Cl. CPC H01L 51/0054; H01L 51/504; C09K 11/06; H01L 51/0067; H01L 51/0058; C07D 251/24; C07D 405/10; H01L 51/0073; C07D 401/10; C07D 405/04; C07D 403/10; H01L 51/0072; C07D 401/04; C07D 401/14; C07D 405/14; C07D 213/16

(57) ABSTRACT

Provided are a compound having a structure represented by Formula I, and an organic light-emitting element, a display panel and a display device including the same. The organic light-emitting element includes an anode, a cathode and an organic thin film disposed between the anode and the cathode; where the organic thin film includes any one or a combination of at least two of a light-emitting layer, an electron transport layer and a hole blocking layer, and includes at least the light-emitting layer, and at least one of the light-emitting layer, the electron transport layer or the hole blocking layer contains at least one of the compounds. The compound has a relatively deep LUMO energy level, a relatively deep HOMO energy level, a relatively high triplet energy level, a high electron mobility, a high T_{g}, and good thermal and chemical stability, and is not easy to crystallize.

### CROSS-REFERENCE TO RELATED APPLICATION(S)

The present disclosure claims priority to a Chinese patent application No. CN 202010524204.0, filed on Jun. 10, 2020 to the CNIPA, the contents of which are incorporated by reference herein in its entirety.

### FIELD

The present disclosure belongs to the field of organic optoelectronic materials and, in particular, relates to a compound, and an organic light-emitting element, a display panel and a display device including the same.

### BACKGROUND

Alq3 is used in traditional electroluminescent elements as an electron transport material. However, Alq3 has a relatively low electron mobility (of about 10⁻⁶ cm²/Vs), so that electron transport and hole transport of the elements are unbalanced. With the productization and practical applications of electroluminescent elements, it is desired to obtain electron transport materials with higher transport efficiency and better performance. Therefore, it is of important practical application values to design and develop stable and efficient electron transport materials and/or electron injection materials that can have both a high electron mobility and a high glass transition temperature.

At present, many electron transport materials commercially available, such as batho-phenanthroline (BPhen), bathocuproine (BCP) and TmPyPB, can generally satisfy the market demand for organic electroluminescent panels, but have a relatively low glass transition temperature T_{g} which is generally less than 85° C. When the elements are operating, generated Joule heat will cause molecular degradation and changes in molecular structure, resulting in low panel efficiency and poor thermal stability. Meanwhile, such symmetrical molecular structures are easy to crystallize after a long period of time. Once the electron transport materials crystallize, the intermolecular charge transition mechanism will differ from the mechanism in normally operated amorphous film, so that electron transport performance decreases, the electron mobility and hole mobility of the entire element are unbalanced, and excitons are formed with greatly reduced efficiency and are concentrated at the interface between the electron transport layer and the light-emitting layer, resulting in a serious decrease in element efficiency and lifetime.

The current researches on organic light-emitting elements are still in a development stage, and there are a few types of good electron transport materials. Therefore, more electron transport materials with better performance are to be developed.

### SUMMARY

In view of defects in the related technics, the present disclosure aims to provide a compound, and an organic light-emitting element, a display panel and a display device including the same. The compound has a relatively deep LUMO energy level, a relatively deep HOMO energy level, a relatively high triplet energy level, a high electron mobility, a high T_{g}, and good thermal and chemical stability, is not easy to crystallize, and may be used in a light-emitting layer, an electron transport layer and/or a hole blocking layer of an organic light-emitting element, to reduce the turn-on voltage of the element and improve light-emitting efficiency and lifetime of the element.

To achieve the object, the present disclosure adopts solutions below.

In a first aspect, the present disclosure provides a compound which has a structure represented by Formula I:

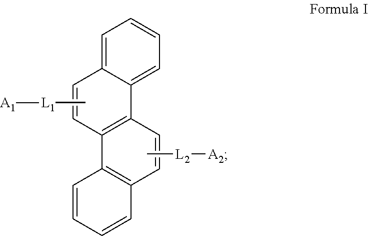

where

L₁ and L₂ are each independently selected from any one of a single bond, substituted or unsubstituted C6 to C40 aryl or substituted or unsubstituted C3 to C40 heteroaryl;

A₁ and A₂ are each independently selected from any one of substituted or unsubstituted C6 to C40 aryl or substituted or unsubstituted C3 to C40 heteroaryl, and at least one of A₁ and A₂ is selected from any one of group

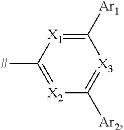

where # represents a position where the group is joined;

where X₁, X₂ and X₃ are each independently a N atom or —CH, and at least one of X₁, X₂ and X₃ is a N atom;

Ar₁ and Ar₂ are each independently selected from any one of substituted or unsubstituted C6 to C40 aryl or substituted or unsubstituted C3 to C40 heteroaryl; and

when a substituent is present in the above groups, the substituent is methyl, ethyl, isopropyl, t-butyl, methoxy, cyano, phenyl, biphenyl, terphenyl, naphthyl, anthryl, phenanthryl, pyridyl, pyrimidyl, triazinyl, carbazolyl, dibenzofuryl or dibenzothienyl.

In a second aspect, the present disclosure provides an organic light-emitting element, including an anode, a cathode and an organic thin film layer disposed between the anode and the cathode; where the organic thin film layer includes one or a combination of at least two of a light-emitting layer, an electron transport layer and a hole blocking layer, and includes at least a light-emitting layer; and

at least one of the light-emitting layer, the electron transport layer and the hole blocking layer contains at least one of the compounds described in the first aspect.

In a third aspect, the present disclosure provides a display panel including the organic light-emitting element described in the second aspect.

In a fourth aspect, the present disclosure provides a display device including the display panel described in the third aspect.

Compared with the related technics, the present disclosure has beneficial effects below.

The compound provided by the present disclosure has a relatively deep LUMO energy level (<−1.70 eV), a relatively deep HOMO energy level(<−5.25 eV), a relatively high triplet energy level (>2.30 eV), a high electron mobility, a high T_{g}, and good thermal and chemical stability, is not easy to crystallize, and may be used in the light-emitting layer, the electron transport layer and/or the hole blocking layer of the organic light-emitting element, to reduce the turn-on voltage of the element and improve the light-emitting efficiency and the lifetime of the element.

### BRIEF DESCRIPTION OF DRAWINGS

FIG. 1 is a structural diagram of an OLED element according to an application example of the present disclosure;

FIG. 2 is a schematic diagram of an organic light-emitting display device according to an application example of the present disclosure.

In the drawings: 1: substrate; 2: anode; 3: hole injection layer; 4: first hole transport layer; 5: second hole transport layer; 6: light-emitting layer; 7: hole blocking layer; 8: electron transport layer; 9: cathode; 10: display of a mobile phone.

### DETAILED DESCRIPTION

The solutions of the present disclosure will be further described below in conjunction with the drawings and examples. The examples described herein are used for a better understanding of the present disclosure and should not be construed as specific limitations to the present disclosure.

In a first aspect, the present disclosure provides a compound which has a structure represented by Formula I:

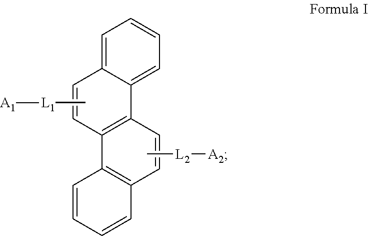

wherein L₁ and L₂ are each independently selected from any one of a single bond, substituted or unsubstituted C6 to C40 (which may be, for example, C6, C8, C10, C12, C15, C18, C21, C24, C28, C30, C34, C36 or C40, etc.) aryl or substituted or unsubstituted C3 to C40 (which may be, for example, C3, C4, C5, C6, C8, C10, C12, C15, C18, C21, C24, C28, C30, C34, C36 or C40, etc.) heteroaryl;

A₁ and A₂ are each independently selected from any one of substituted or unsubstituted C6 to C40 (which may be, for example, C6, C8, C10, C12, C15, C18, C21, C24, C28, C30, C34, C36 or C40, etc.) aryl or substituted or unsubstituted C3 to C40 (which may be, for example, C3, C4, C5, C6, C8, C10, C12, C15, C18, C21, C24, C28, C30, C34, C36 or C40, etc.) heteroaryl, and at least one of A₁ and A₂ is selected from any one of group

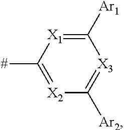

where # represents a position where the group is joined;

X₁, X₂ and X₃ are each independently a N atom or —CH, and at least one of X₁, X₂ and X₃ is a N atom;

Ar₁ and Ar₂ are each independently selected from any one of substituted or unsubstituted C6 to C40 (which may be, for example, C6, C8, C10, C12, C15, C18, C21, C24, C28, C30, C34, C36 or C40, etc.) aryl or substituted or unsubstituted C3 to C40 (which may be, for example, C3, C4, C5, C6, C8, C10, C12, C15, C18, C21, C24, C28, C30, C34, C36 or C40, etc.) heteroaryl; and

when a substituent is present in the above groups, the substituent is methyl, ethyl, isopropyl, t-butyl, methoxy, cyano, phenyl, biphenyl, terphenyl, naphthyl, anthryl, phenanthryl, pyridyl, pyrimidyl, triazinyl, carbazolyl, dibenzofuryl or dibenzothienyl.

The compound provided by the present disclosure has a core structure of chrysene connected to a nitrogen-containing heterocyclic ring

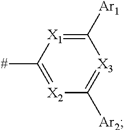

its molecule has a large conjugated structure so that the compound has a relatively high electron mobility, which helps to increase the rate at which excitons are generated; has a relatively deep LUMO energy level and a small electron injection barrier, which is advantageous for the injection of electrons; has a relatively deep HOMO energy level, which helps to block holes; has a relatively high triplet energy level, which can effectively block excitons and confine the excitons in a light-emitting layer; has a high T_{g} (glass transition temperature) and good thermal and chemical stability, which helps to reduce the effect of Joule heat generated by an organic light-emitting element including the compound during working on lifetime and efficiency of the element; and is not easy to crystallize, which helps to reduce light scattering and degradation or decrease in element efficiency induced by crystallization. The compound provided by the present disclosure can be used in a light-emitting layer, an electron transport layer and/or a hole blocking layer of an organic light-emitting element, to reduce the turn-on voltage of the organic light-emitting element and improve light-emitting efficiency and the lifetime.

It is to be noted that the LUMO energy level and the HOMO energy level of the compound provided by the present disclosure have negative values, a “high” or “shallow” HOMO energy level or LUMO energy level in the present disclosure means a large numeral value but a small absolute value; and a “low” or “deep” HOMO energy level or LUMO energy level means a small numeral value but a large absolute value.

In an embodiment of the present disclosure, the compound has a structure represented by Formula II:

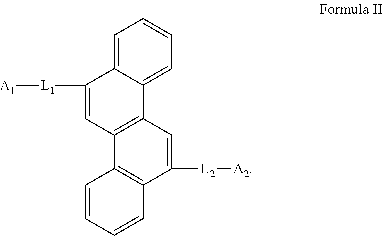

In an embodiment of the present disclosure, L₁ and L₂ are each independently selected from any one of a single bond, phenylene, biphenylene, naphthylene, anthrylene, furylene, thienylene, pyridylene, pyrimidylene, triazinylene or fluorenylene.

In an embodiment of the present disclosure, A₁ and A₂ are each independently selected from any one of phenyl, naphthyl, anthryl, pyridyl, pyrimidyl, triazinyl, dibenzofuryl, dibenzothienyl, carbazolyl, fluorenyl, spirofluorenyl or

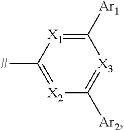

and at least one of A₁ and A₂ is selected from any one of

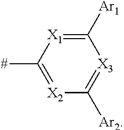

In an embodiment of the present disclosure, Ar₁ and Ar₂ are each independently selected from any one of phenyl, biphenyl, terphenyl, naphthyl, anthryl, phenanthryl, pyridyl, pyrimidyl, triazinyl, dibenzofuryl, dibenzothienyl, carbazolyl, fluorenyl or spirofluorenyl.

In an embodiment of the present disclosure, the group

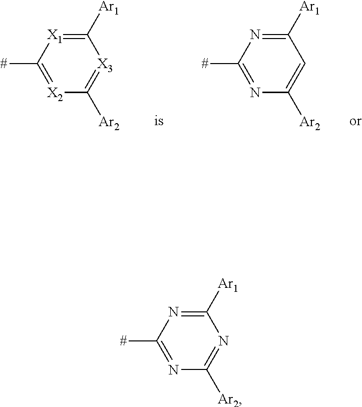

where # represents the position where the group is joined.

In the present disclosure, the group

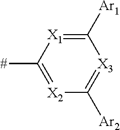

is most preferably

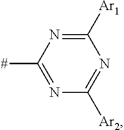

and such compounds, compared with compounds containing

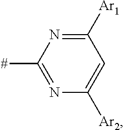

have better stability and deeper LOMO energy levels, are more advantageous for the injection of electrons, and can be better complexed with a metal dopant of the electron transport layer, improving efficiency of organic light-emitting elements.

In an embodiment of the present disclosure, the compound has a structure represented by Formula III or Formula IV:

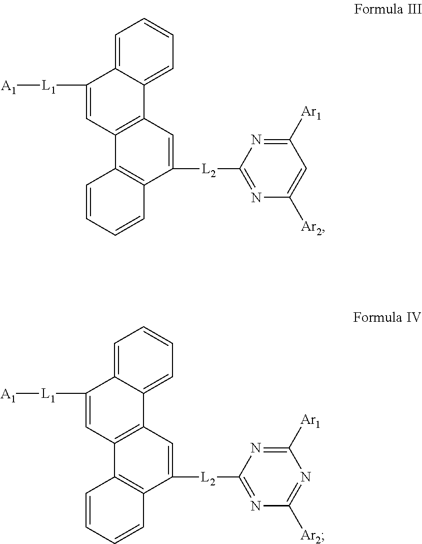

wherein L₁ and L₂ are each independently selected from any one of a single bond, phenylene, biphenylene, naphthylene, anthrylene, furylene, thienylene, pyridylene, pyrimidylene, triazinylene or fluorenylene;

A₁ is selected from any one of phenyl, naphthyl, anthryl, pyridyl, pyrimidyl, triazinyl, dibenzofuryl, dibenzothienyl, carbazolyl, fluorenyl, spirofluorenyl,

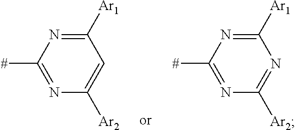

Ar₁ and Ar₂ are each independently selected from any one of phenyl, biphenyl, terphenyl, naphthyl, anthryl, phenanthryl, pyridyl, pyrimidyl, triazinyl, dibenzofuryl, dibenzothienyl, carbazolyl, fluorenyl or spirofluorenyl.

In an embodiment of the present disclosure, the compound has a structure represented by Formula IV;

wherein L₁ and L₂ are each independently a single bond, phenylene or naphthylene;

A₁ is selected from any one of phenyl, naphthyl, pyridyl, dibenzofuryl, dibenzothienyl, carbazolyl, fluorenyl or

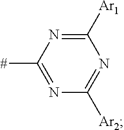

and

Ar₁ and Ar₂ are each independently selected from any one of phenyl, biphenyl, pyridyl, dibenzofuryl, dibenzothienyl, carbazolyl or fluorenyl.

In an embodiment of the present disclosure, the compound is selected from any one of the following compounds H1 to H81:

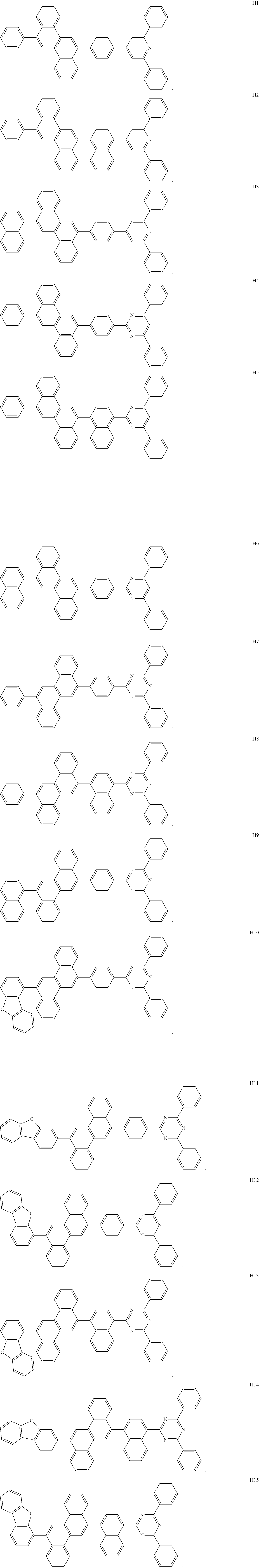

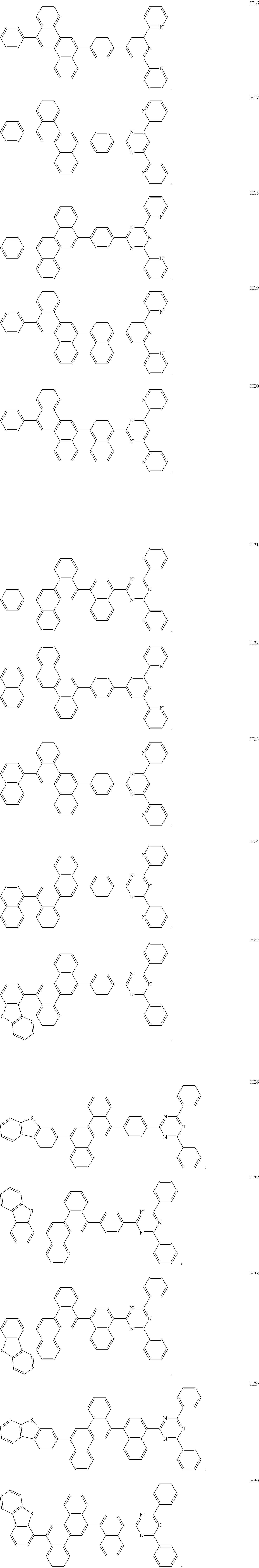

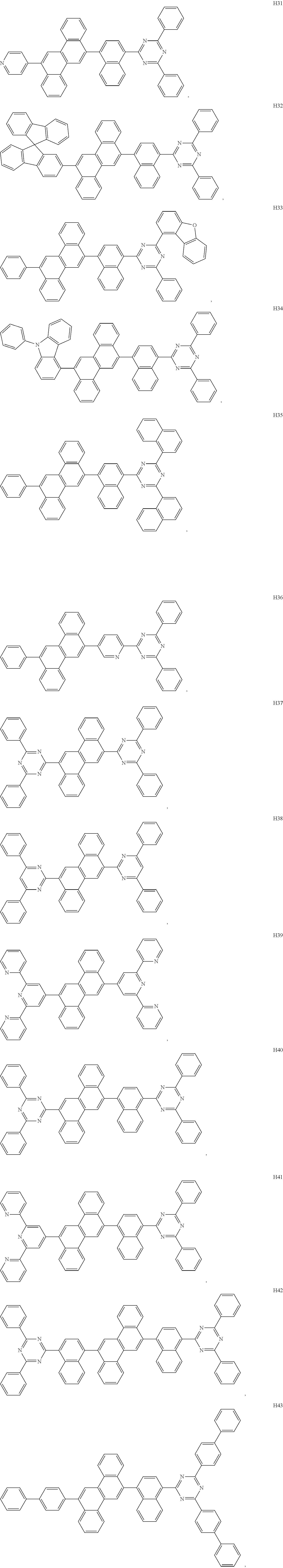

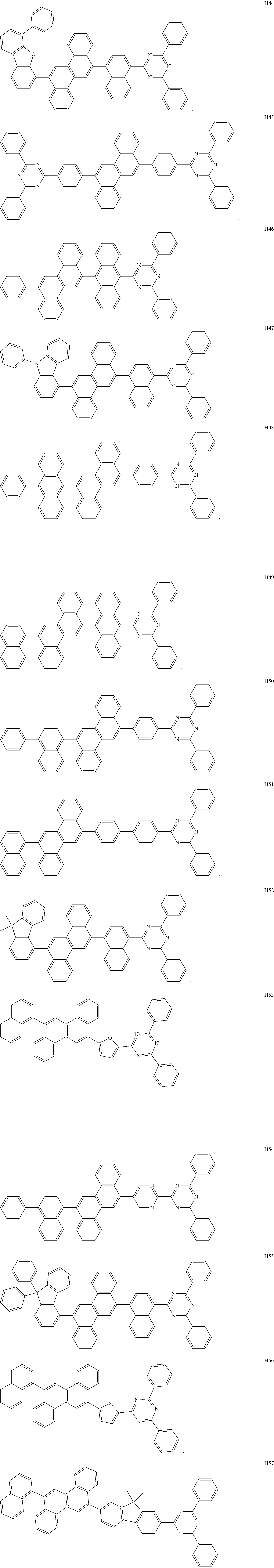

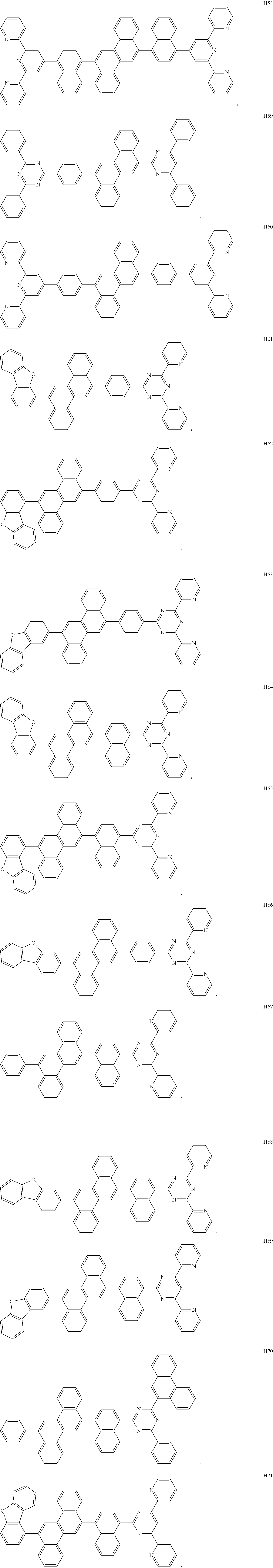

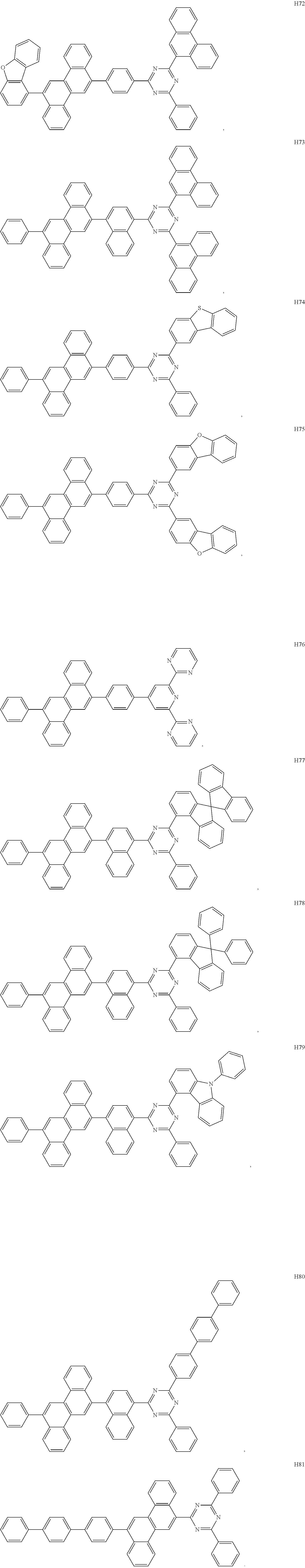

In a second aspect, the present disclosure provides an organic light-emitting element (an OLED element), including an anode, a cathode and an organic thin film layer disposed between the anode and the cathode; where the organic thin film layer includes one or a combination of at least two of a light-emitting layer, an electron transport layer and a hole blocking layer, and includes at least a light-emitting layer;

wherein at least one of the light-emitting layer, the electron transport layer and the hole blocking layer contains at least one of the compounds described in the first aspect.

In an embodiment of the present disclosure, the organic thin film layer further includes any one or a combination of at least two of a hole injection layer, a hole transport layer, an electron blocking layer and an electron injection layer.

In an embodiment of the present disclosure, the light-emitting layer includes a host material and a light-emitting material, where the host material of the light-emitting layer includes any one or a combination of at least two of the compounds described in the first aspect.

In an embodiment of the present disclosure, a material of the electron transport layer is selected from any one or a combination of at least two of the compounds described in the first aspect;

alternatively, the electron transport layer includes a host material and a guest material, where the host material of the electron transport layer is selected from any one or a combination of at least two of the compounds described in the first aspect.

In an embodiment of the present disclosure, a material of the hole blocking layer is selected from any one or a combination of at least two of the compounds described in the first aspect, and the light-emitting material of the light-emitting layer has a lowest triplet energy level which is lower than the lowest triplet energy level of the compound.

In a third aspect, the present disclosure provides a display panel including the organic light-emitting element described in the second aspect.

In a fourth aspect, the present disclosure provides a display device including the display panel described in the third aspect.

The examples of the present disclosure exemplarily provide the following compounds and preparation methods thereof, and adopt these compounds to exemplarily prepare organic light-emitting elements. The examples described herein are used for a better understanding of the present disclosure and should not be construed as specific limitations to the present disclosure.

### Preparation Example 1

A compound H2 is prepared by using the following specific steps:

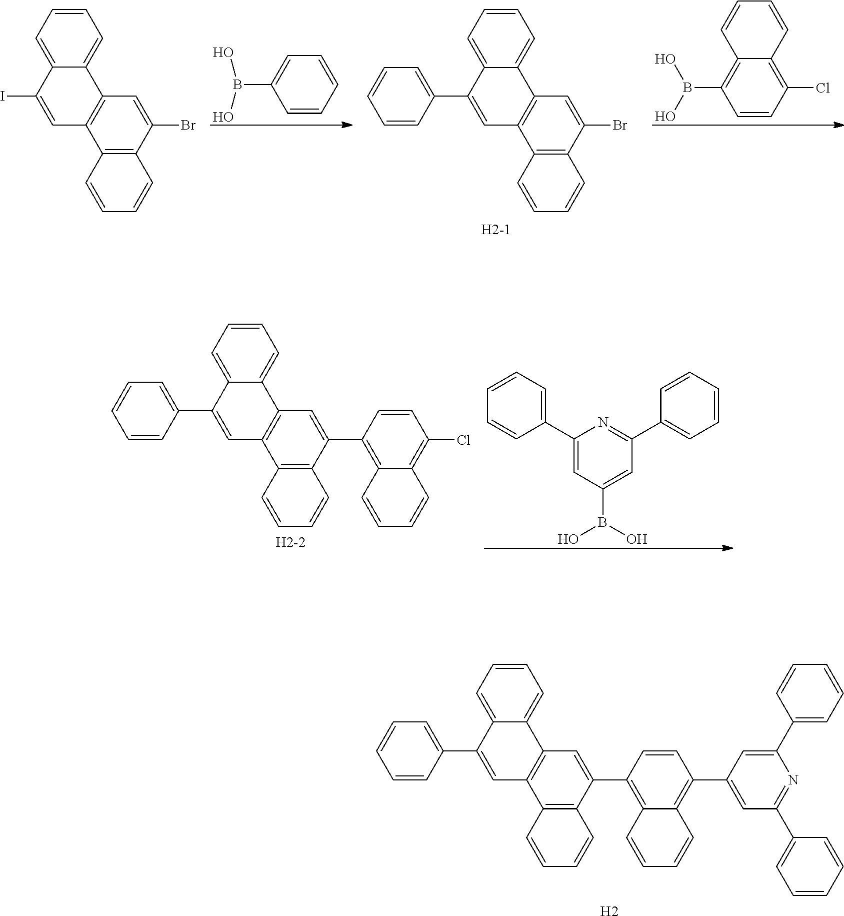

In a 250 mL round-bottom flask, 6-iodo-12-bromo-chrysene (12 mmol), phenylboric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H2-1.

Characterization results of the intermediate product H2-1:

Elemental analysis result: C24H15Br, calcd.: C 75.21, H 3.94, Br 20.85; found: C 75.21, H 3.95, Br 20.84.

ESI-MS (m/z) (M+) obtained through liquid chromatography-mass spectrometry analysis: calcd.: 382.04; found: 382.28.

In a 250 mL round-bottom flask, the intermediate product H2-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H2-2.

Characterization results of the intermediate product H2-2:

Elemental analysis result: C34H21Cl, calcd.: C 87.82, H 4.55, Cl 7.62; found: C 87.82, H 4.54, Cl 7.63.

ESI-MS (m/z) (M+) obtained through liquid chromatography-mass spectrometry analysis: calcd.: 464.13; found: 464.98.

In a 250 mL round-bottom flask, the intermediate product H2-2 (12 mmol), (2,6-diphenyl-2-pyridyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H2.

Characterization results of the compound H2:

Elemental analysis result: C51H33N, calcd.: C 92.84, H 5.04, N 2.12; found: C 92.84, H 5.05, N 2.11.

ESI-MS (m/z) (M+) obtained through liquid chromatography-mass spectrometry analysis: calcd.: 659.26; found: 659.81.

### Preparation Example 2

A compound H8 is prepared by using the following specific steps:

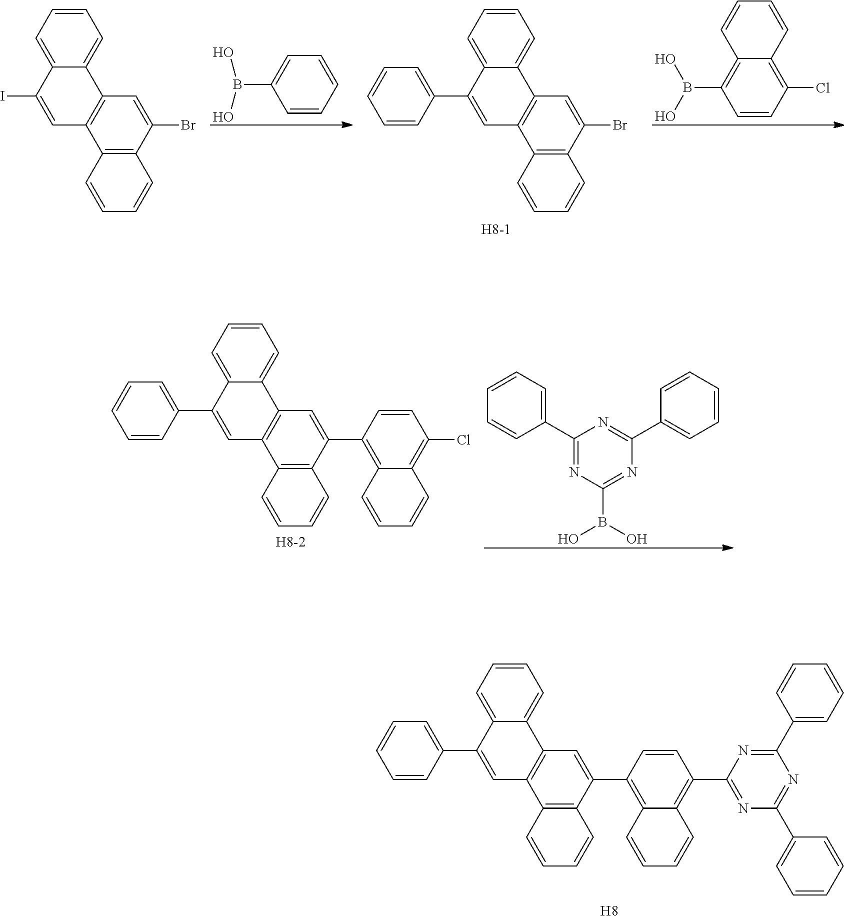

In a 250 mL round-bottom flask, 6-iodo-12-bromo-chrysene (12 mmol), phenylboric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H8-1.

Characterization results of the intermediate product H8-1:

Elemental analysis result: C24H15Br, calcd.: C 75.21, H 3.94, Br 20.85; found: C 75.21, H 3.95, Br 20.84.

ESI-MS (m/z) (M+) obtained through liquid chromatography-mass spectrometry analysis: calcd.: 382.04; found: 383.28.

In a 250 mL round-bottom flask, the intermediate product H8-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H8-2.

Characterization results of the intermediate product H8-2:

Elemental analysis result: C34H21Cl, calcd.: C 87.82, H 4.55, Cl 7.62; found: C 87.82, H 4.54, Cl 7.63.

ESI-MS (m/z) (M+) obtained through liquid chromatography-mass spectrometry analysis: calcd.: 464.13; found: 464.98.

In a 250 mL round-bottom flask, the intermediate product H8-2 (12 mmol), 2-boric acid-4,6-diphenyl-triazine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H8.

Characterization results of the compound H8:

Elemental analysis result: C49H31N3, calcd.: C 88.93, H 4.72, N 6.35; found: C 88.95, H 4.74, N 6.31.

ESI-MS (m/z) (M+) obtained through liquid chromatography-mass spectrometry analysis: calcd.: 661.25; found: 661.79.

### Preparation Example 3

A compound H15 is prepared by using the following specific steps:

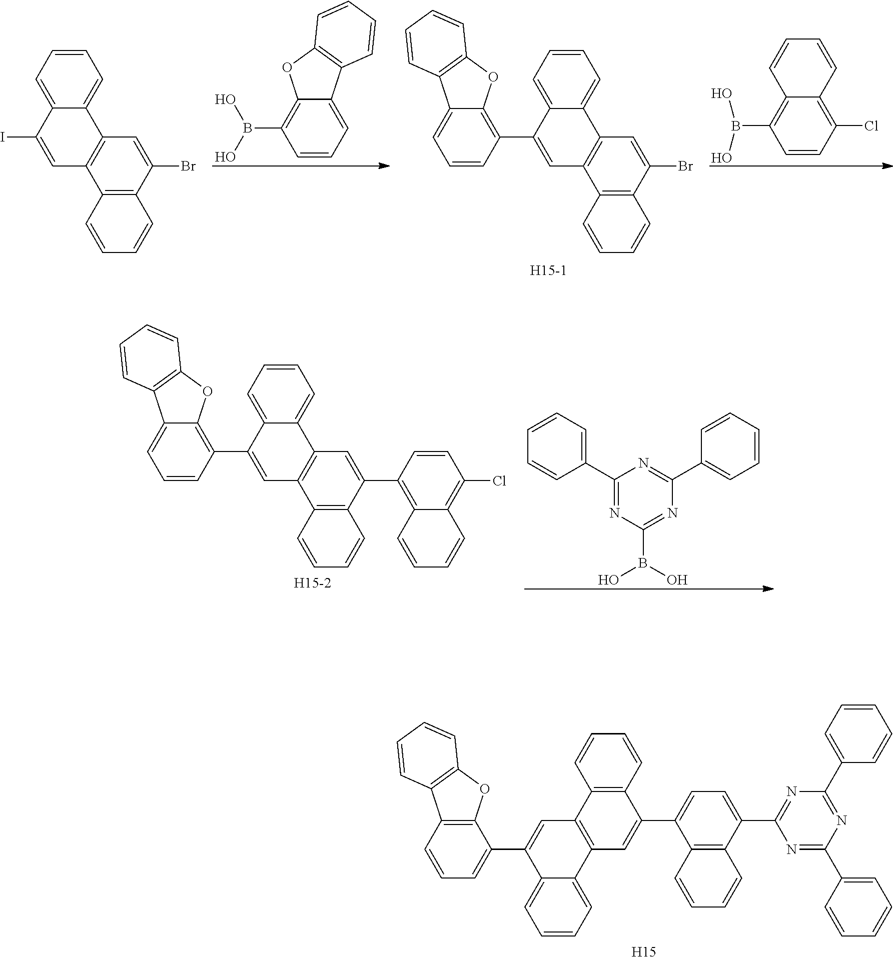

In a 250 mL round-bottom flask, 6-iodo-12-bromo-chrysene (12 mmol), (4-dibenzofuran)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H15-1.

Characterization results of the intermediate product H15-1:

Elemental analysis result: C30H17BrO, calcd.: C 76.12, H 3.62, Br 16.88, O 3.38; found: C 76.12, H 3.64, Br 16.87, O 3.37.

ESI-MS (m/z) (M+) obtained through liquid chromatography-mass spectrometry analysis: calcd.: 472.05; found: 473.36.

In a 250 mL round-bottom flask, the intermediate product H15-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H15-2.

Characterization results of the intermediate product H15-2:

Elemental analysis result: C40H23ClO, calcd.: C 86.55, H 4.18, Cl 6.39, O 2.88; found: C 86.55, H 4.17, Cl 6.40, O 2.88.

ESI-MS (m/z) (M+) obtained through liquid chromatography-mass spectrometry analysis: calcd.: 554.14; found: 555.06.

In a 250 mL round-bottom flask, the intermediate product H15-2 (12 mmol), 2-boric acid-4,6-diphenyl-triazine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H15.

Characterization results of the compound H15:

Elemental analysis result: C55H33N3O, calcd.: C 87.86, H 4.42, N 5.59, O 2.13; found: C 87.88, H 4.45, N 5.57, O 2.10.

ESI-MS (m/z) (M+) obtained through liquid chromatography-mass spectrometry analysis: calcd.: 751.26; found: 751.87.

### Preparation Example 4

A compound H31 is prepared by using the following specific steps:

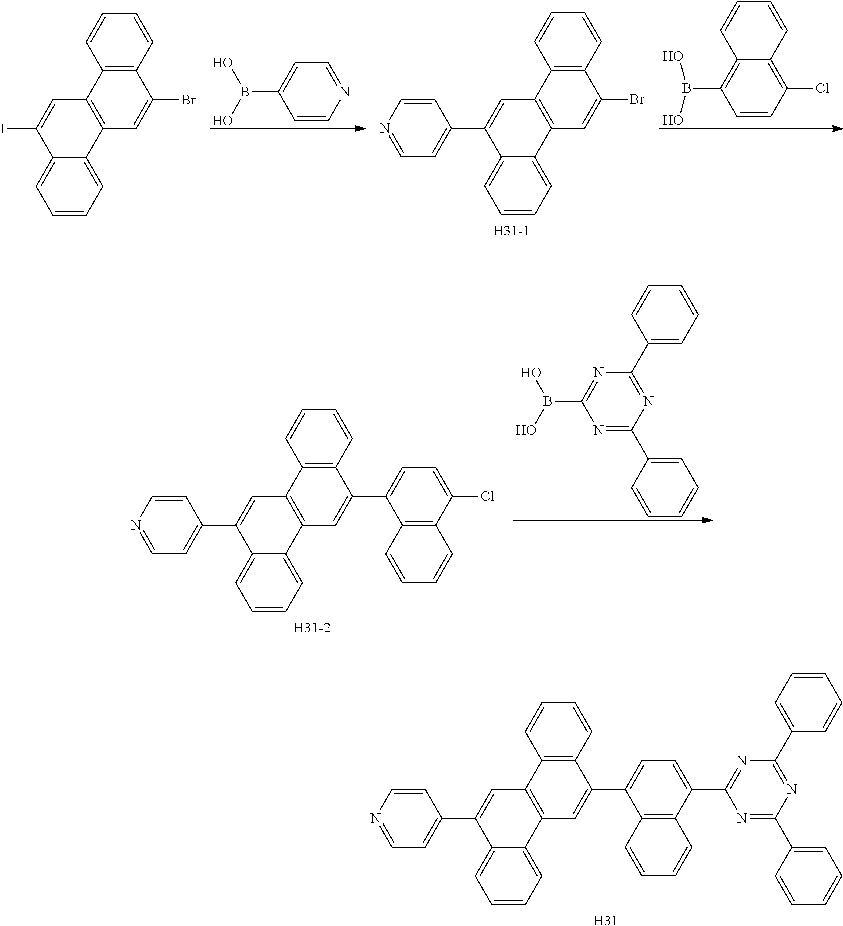

In a 250 mL round-bottom flask, 6-iodo-12-bromo-chrysene (12 mmol), pyridylboric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H31-1.

Characterization results of the intermediate product H31-1:

Elemental analysis result: C23H14BrN, calcd.: C 71.89, H 3.67, Br 20.79, N 3.65; found: C 71.89, H 3.69, Br 20.78, N 3.66.

ESI-MS (m/z) (M+) obtained through liquid chromatography-mass spectrometry analysis: calcd.: 383.03; found: 384.27.

In a 250 mL round-bottom flask, the intermediate product H31-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H31-2.

Characterization results of the intermediate product H31-2:

Elemental analysis result: C33H20ClN, calcd.: C 85.06, H 4.33, Cl 7.61, N 3.01; found: C 85.06, H 4.31, Cl 7.61, N 3.03.

ESI-MS (m/z) (M+) obtained through liquid chromatography-mass spectrometry analysis: calcd.: 465.13; found: 465.97.

In a 250 mL round-bottom flask, the intermediate product H31-2 (12 mmol), 2-boric acid-4,6-diphenyl-triazine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H31.

Characterization results of the compound H31:

Elemental analysis result: C48H30N4, calcd.: C 86.98, H 4.56, N 8.45; found: C 86.98, H 4.58, N 8.43.

ESI-MS (m/z) (M+) obtained through liquid chromatography-mass spectrometry analysis: calcd.: 662.25; found: 662.78.

### Preparation Example 5

A compound H33 is prepared by using the following specific steps:

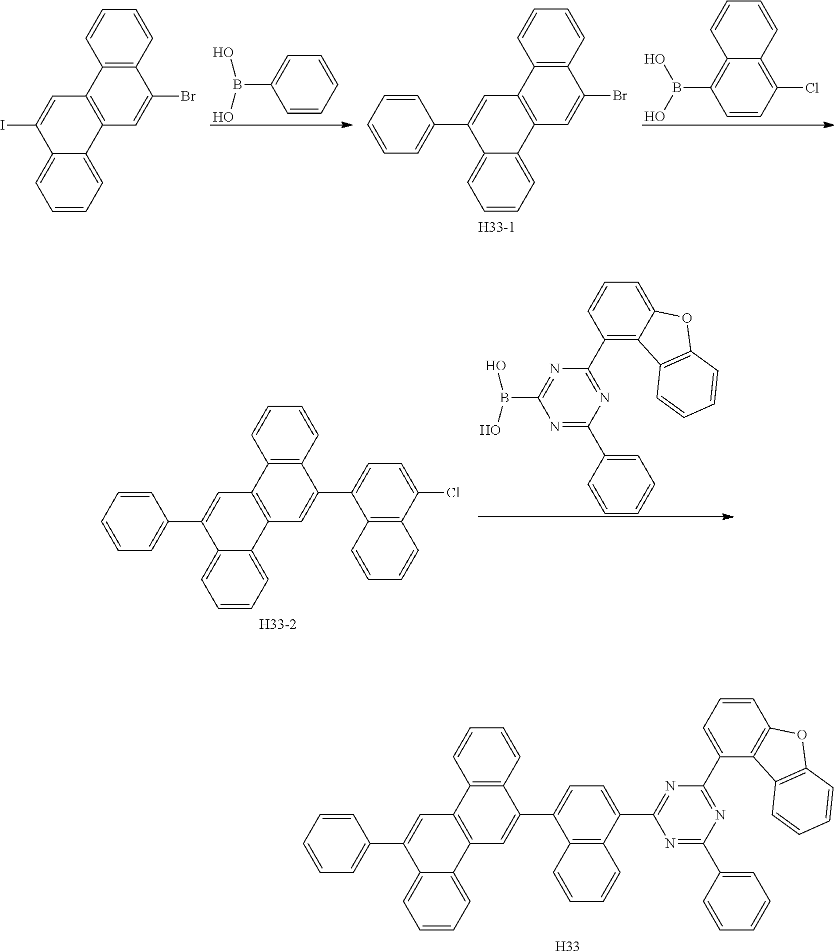

In a 250 mL round-bottom flask, 6-iodo-12-bromo-chrysene (12 mmol), phenylboric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H33-1.

Characterization results of the intermediate product H33-1:

Elemental analysis result: C24H15Br, calcd.: C 75.21, H 3.94, Br 20.85; found: C 75.21, H 3.95, Br 20.84.

ESI-MS (m/z) (M+) obtained through liquid chromatography-mass spectrometry analysis: calcd.: 382.04; found: 383.28.

In a 250 mL round-bottom flask, the intermediate product H33-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H33-2.

Characterization results of the intermediate product H33-2:

Elemental analysis result: C34H21Cl, calcd.: C 87.82, H 4.55, Cl 7.62; found: C 87.82, H 4.54, Cl 7.63.

ESI-MS (m/z) (M+) obtained through liquid chromatography-mass spectrometry analysis: calcd.: 464.13; found: 464.98.

In a 250 mL round-bottom flask, the intermediate product H33-2 (12 mmol), 2-boric acid-4-(1-dibenzofuran)-6-phenyl-triazine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H33.

Characterization results of the compound H33:

Elemental analysis result: C55H33N3O, calcd.: C 87.86, H 4.42, N 5.59, O 2.13; found: C 87.81, H 4.45, N 5.58, O 2.16.

ESI-MS (m/z) (M+) obtained through liquid chromatography-mass spectrometry analysis: calcd.: 751.26; found: 751.87.

### Preparation Example 6

A compound H34 is prepared by using the following specific steps:

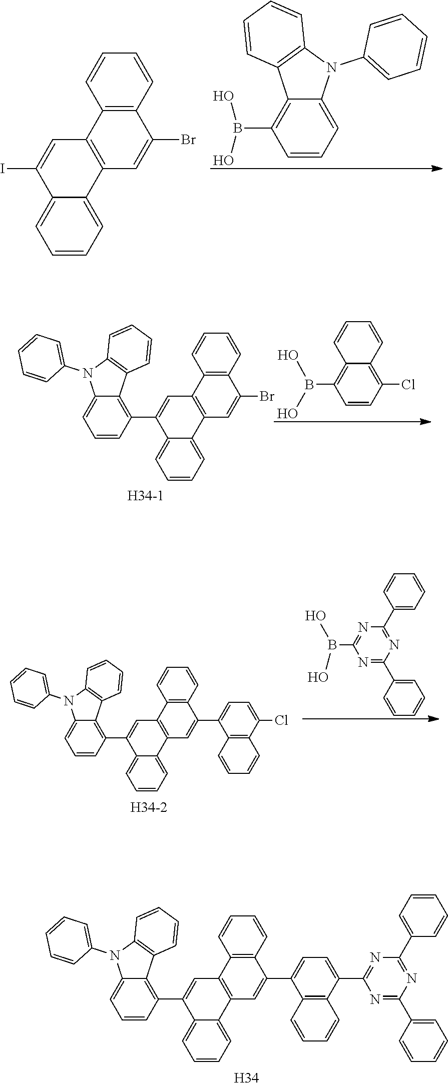

In a 250 mL round-bottom flask, 6-iodo-12-bromo-chrysene (12 mmol), 9-phenylcarbazolylboric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H34-1.

Characterization results of the intermediate product H34-1:

Elemental analysis result: C36H22BrN, calcd.: C 78.83, H 4.04, Br 14.57, N 2.55; found: C 78.83, H 4.01, Br 14.58, N 2.57.

ESI-MS (m/z) (M+) obtained through liquid chromatography-mass spectrometry analysis: calcd.: 547.09; found: 548.47.

In a 250 mL round-bottom flask, the intermediate product H34-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H34-2.

Characterization results of the intermediate product H34-2:

Elemental analysis result: C46H28C1N, calcd.: C 87.67, H 4.48, Cl 5.63, N 2.22; found: C 87.67, H 4.45, Cl 5.65, N 2.23.

ESI-MS (m/z) (M+) obtained through liquid chromatography-mass spectrometry analysis: calcd.: 629.19; found: 630.17.

In a 250 mL round-bottom flask, the intermediate product H34-2 (12 mmol), 2-boric acid-4,6-diphenyl-triazine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H34.

Characterization results of the compound H34:

Elemental analysis result: C61H38N4, calcd.: C 88.59, H 4.63, N 6.77; found: C 88.59, H 4.65, N 6.75.

ESI-MS (m/z) (M+) obtained through liquid chromatography-mass spectrometry analysis: calcd.: 826.31; found: 826.98.

### Preparation Example 7

A compound H36 is prepared by using the following specific steps:

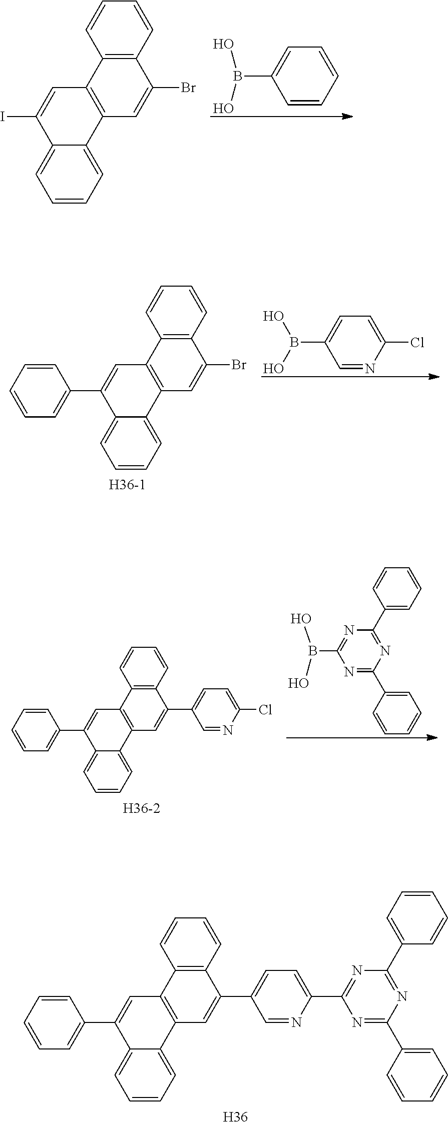

In a 250 mL round-bottom flask, 6-iodo-12-bromo-chrysene (12 mmol), phenylboric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H36-1.

Characterization results of the intermediate product H36-1:

Elemental analysis result: C24H15Br, calcd.: C 75.21, H 3.94, Br 20.85; found: C 75.21, H 3.95, Br 20.84.

ESI-MS (m/z) (M+) obtained through liquid chromatography-mass spectrometry analysis: calcd.: 382.04; found: 383.28.

In a 250 mL round-bottom flask, the intermediate product H36-1 (12 mmol), (6-chloropyridyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H36-2.

Characterization results of the intermediate product H36-2:

Elemental analysis result: C29H18ClN, calcd.: C 83.75, H 4.36, Cl 8.52, N: 3.37; found: C 83.75, H 4.38, Cl 8.51, N 3.36.

ESI-MS (m/z) (M+) obtained through liquid chromatography-mass spectrometry analysis: calcd.: 415.11; found: 415.91.

In a 250 mL round-bottom flask, the intermediate product H36-2 (12 mmol), 2-boric acid-4,6-diphenyl-triazine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H36.

Characterization results of the compound H36:

Elemental analysis result: C44H28N4, calcd.: C 86.25, H 4.61, N 9.14; found: C 86.25, H 4.62, N 9.13.

ESI-MS (m/z) (M+) obtained through liquid chromatography-mass spectrometry analysis: calcd.: 612.23; found: 612.72.

### Preparation Example 8

A compound H37 is prepared by using the following specific steps:

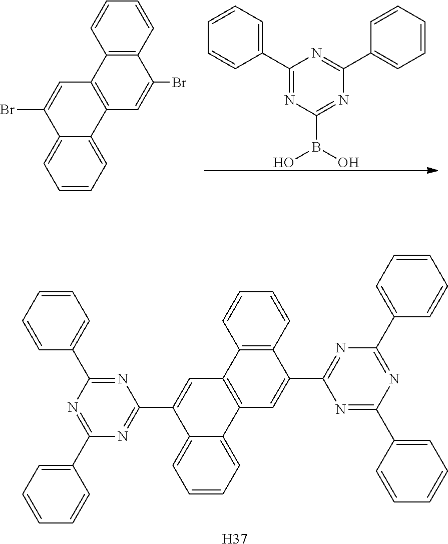

In a 250 mL round-bottom flask, 6,12-dibromo-chrysene (12 mmol), 2-boric acid-4,6-diphenyl-triazine (25 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H37.

Characterization results of the compound H37:

Elemental analysis result: C48H30N6, calcd.: C 83.46, H 4.38, N 12.17; found: C 83.42, H 4.39, N 12.20.

ESI-MS (m/z) (M+) obtained through liquid chromatography-mass spectrometry analysis: calcd.: 690.25; found: 690.79.

### Preparation Example 9

A compound H41 is prepared by using the following specific steps:

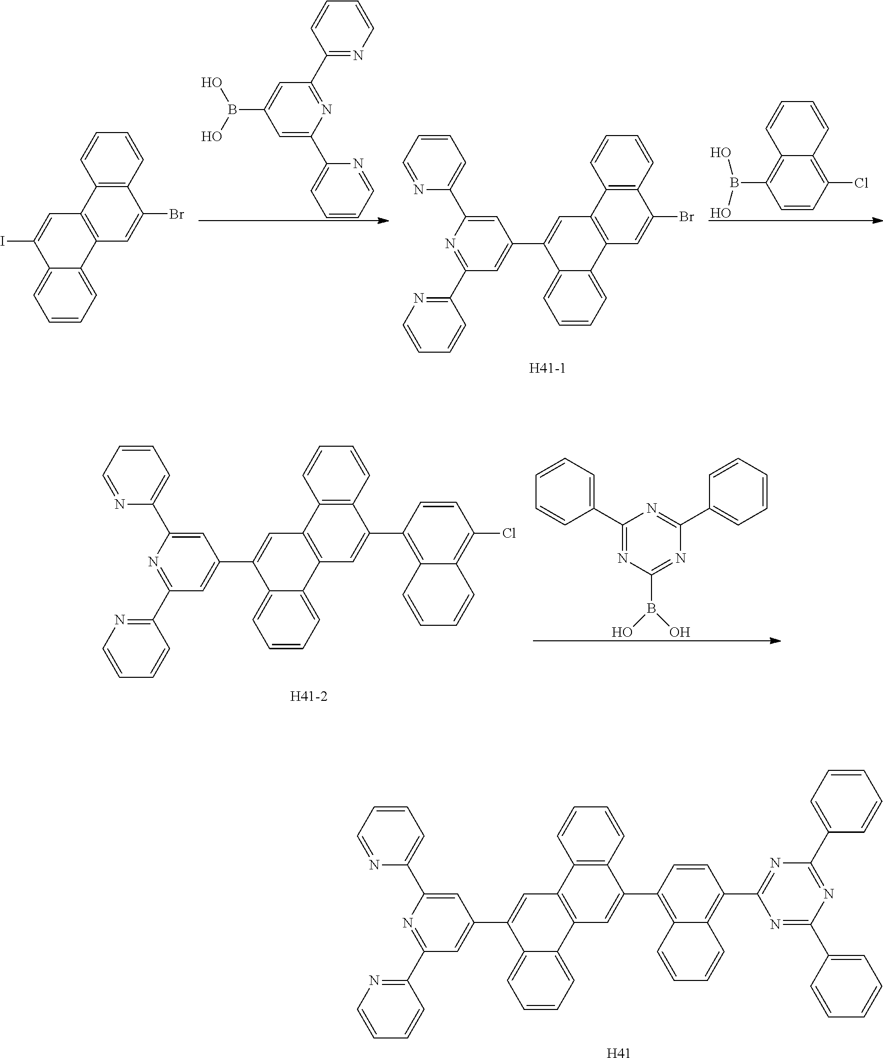

In a 250 mL round-bottom flask, 6-iodo-12-bromo-chrysene (12 mmol), 2-boric acid-4,6-dipyridyl-pyridine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H41-1.

Characterization results of the intermediate product H41-1:

Elemental analysis result: C33H20BrN3, calcd.: C 73.61, H 3.74, Br 14.84, N 7.80; found: C 73.61, H 3.75, Br 14.84, N 7.79.

ESI-MS (m/z) (M+) obtained through liquid chromatography-mass spectrometry analysis: calcd.: 537.08; found: 538.44.

In a 250 mL round-bottom flask, the intermediate product H41-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H41-2.

Characterization results of the intermediate product H41-2:

Elemental analysis result: C43H26ClN3, calcd.: C 83.28, H 4.23, Cl 5.72, N 6.78; found: C 83.28, H 4.24, Cl 5.72, N 6.77.

ESI-MS (m/z) (M+) obtained through liquid chromatography-mass spectrometry analysis: calcd.: 619.18; found: 620.14.

In a 250 mL round-bottom flask, the intermediate product H41-2 (12 mmol), 2-boric acid-4,6-diphenyl-triazine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H41.

Characterization results of the compound H41:

Elemental analysis result: C58H36N6, calcd.: C 85.27, H 4.44, N 10.29; found: C 85.22, H 4.46, N 10.32.

ESI-MS (m/z) (M+) obtained through liquid chromatography-mass spectrometry analysis: calcd.: 816.30; found: 816.95.

### Preparation Example 10

A compound H43 is prepared by using the following specific steps:

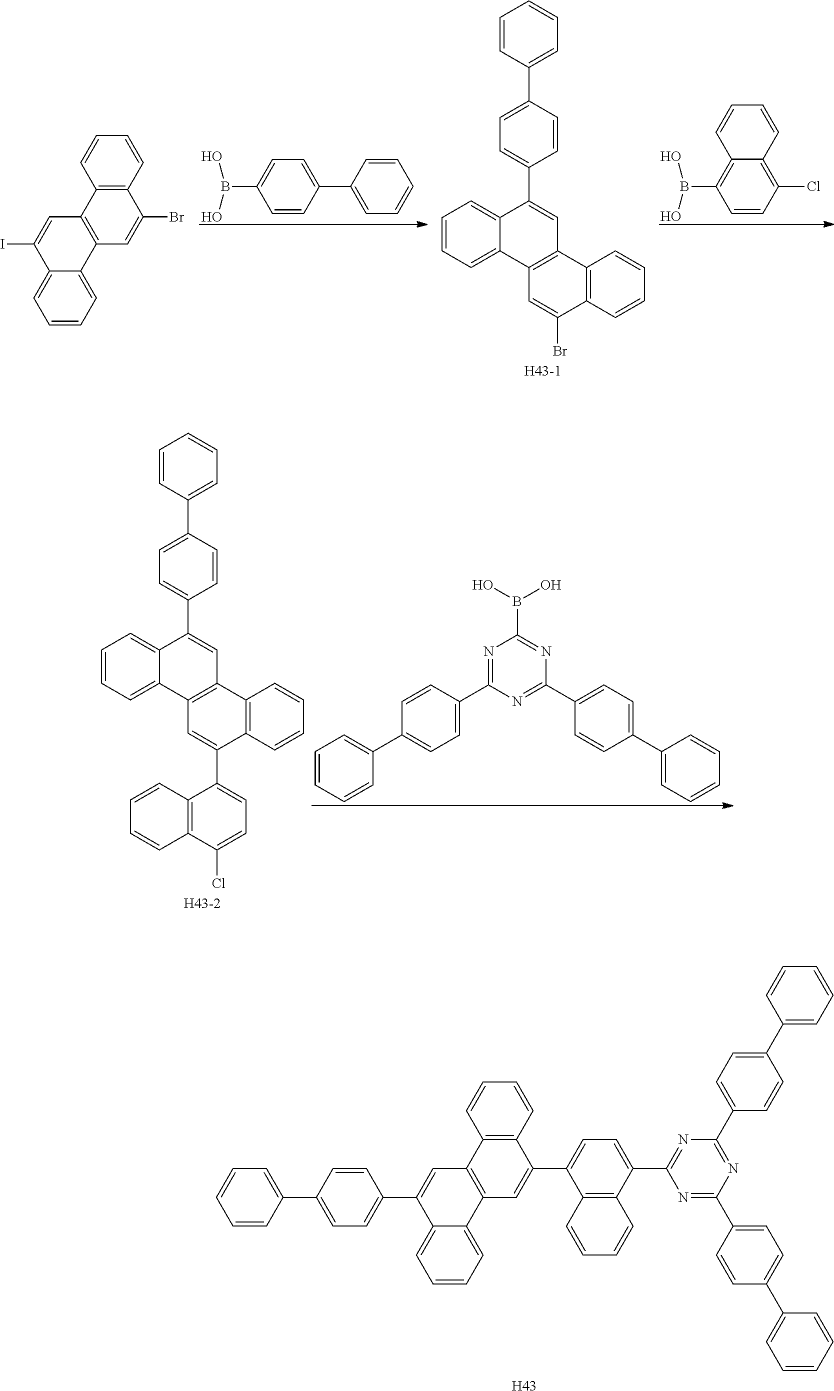

In a 250 mL round-bottom flask, 6-iodo-12-bromo-chrysene (12 mmol), biphenylboric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H43-1.

Characterization results of the intermediate product H43-1:

Elemental analysis result: C30H19Br, calcd.: C 78.44, H 4.17, Br 17.39; found: C 78.43, H 4.19, Br 17.38.

ESI-MS (m/z) (M+) obtained through liquid chromatography-mass spectrometry analysis: calcd.: 458.07; found: 459.38.

In a 250 mL round-bottom flask, the intermediate product H43-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H43-2.

Characterization results of the intermediate product H43-2:

Elemental analysis result: C40H25Cl, calcd.: C 88.79, H 4.66, Cl 6.55; found: C 88.79, H 4.67, Cl 6.54.

ESI-MS (m/z) (M+) obtained through liquid chromatography-mass spectrometry analysis: calcd.: 540.16; found: 541.08.

In a 250 mL round-bottom flask, the intermediate product H43-2 (12 mmol), 2-boric acid-4,6-dibiphenyl-triazine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H43.

Characterization results of the compound H43:

Elemental analysis result: C67H43N3, calcd.: C 90.41, H 4.87, N 4.72; found: C 90.37, H 4.88, N 4.75.

ESI-MS (m/z) (M+) obtained through liquid chromatography-mass spectrometry analysis: calcd.: 889.35; found: 890.08.

### Preparation Example 11

A compound H71 is prepared by using the following specific steps:

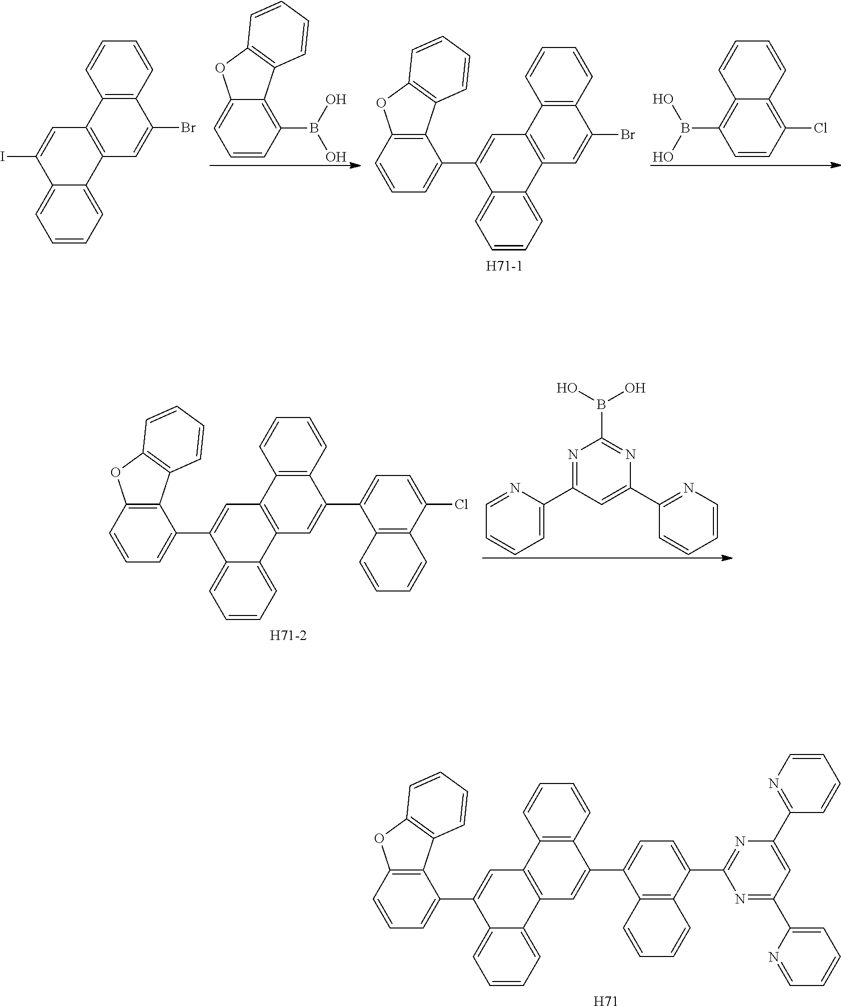

In a 250 mL round-bottom flask, 6-iodo-12-bromo-chrysene (12 mmol), (1-dibenzofuran)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H71-1.

Characterization results of the intermediate product H71-1:

Elemental analysis result: C30H17BrO, calcd.: C 76.12, H 3.62, Br 16.88, O 3.38; found: C 76.12, H 3.60, Br 16.89, O 3.39.

ESI-MS (m/z) (M+) obtained through liquid chromatography-mass spectrometry analysis: calcd.: 472.05; found: 473.36.

In a 250 mL round-bottom flask, the intermediate product H71-1 (12 mmol), (4-chloronaphthyl)boric acid (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain an intermediate product H71-2.

Characterization results of the intermediate product H71-2:

Elemental analysis result: C40H23ClO, calcd.: C 86.55, H 4.18, Cl 6.39, O 2.88; found: C 86.55, H 4.16, Cl 6.40, O 2.89.

ESI-MS (m/z) (M+) obtained through liquid chromatography-mass spectrometry analysis: calcd.: 554.14; found: 555.06.

In a 250 mL round-bottom flask, the intermediate product H71-2 (12 mmol), 2-boric acid-4,6-dipyridyl-pyrimidine (12 mmol) and Na₂CO₃ (80 mmol) were separately added to a mixed solvent of toluene/absolute ethanol (EtOH)/H₂O (75/25/50 in mL) to obtain a mixed solution. Then Pd(PPh₃)₄ (0.48 mmol) was added to the above mixed solution and refluxed for 20 h in a nitrogen atmosphere. The obtained intermediate was cooled to room temperature, added into water, filtered through a pad of Celite and extracted with dichloromethane, then washed with water, dried with anhydrous magnesium sulfate, filtered and evaporated to obtain a crude product. The crude product was purified through silica gel column chromatography to obtain a final product H71.

Characterization results of the compound H71:

Elemental analysis result: C54H32N4O, calcd.: C 86.15, H 4.28, N 7.44, O 2.13; found: C 86.15, H 4.30, N 7.43, O 2.12.

ESI-MS (m/z) (M+) obtained through liquid chromatography-mass spectrometry analysis: calcd.: 752.26; found: 752.86.

Simulated calculations of energy levels of compounds

By use of the density functional theory (DFT), the distribution of molecular frontier orbitals, HOMO and LUMO, was optimized and calculated for the organic compounds provided in the examples of the present disclosure and comparative compound 1

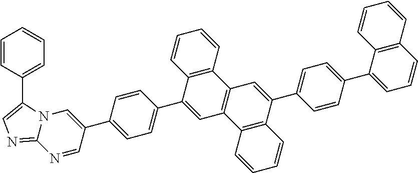 [SMILES: c1ccc(-c2cnc3ncc(-c4ccc(-c5cc6c7ccccc7c(-c7ccc(-c8cccc9ccccc89)cc7)cc6c6ccccc56)cc4)cn23)cc1]

using a Guassian 09 package (Guassian Inc.) at a B3LYP/6-31G(d) calculation level. Meanwhile, based on the time-dependent density functional theory (TDDFT), the lowest triplet energy level E_{T1} of molecules of each compound was simulated and calculated. Results are shown in Table 1.

TABLE 1

| Compound | HOMO (eV) | LUMO (eV) | Eg (eV) | E_{T1} (eV) |
|---|---|---|---|---|
| H2 | −5.31 | −1.74 | 3.57 | 2.38 |
| H8 | −5.39 | −1.90 | 3.49 | 2.36 |
| H15 | −5.29 | −1.89 | 3.40 | 2.32 |
| H31 | −5.33 | −1.91 | 3.42 | 2.32 |
| H33 | −5.35 | −1.90 | 3.45 | 2.35 |
| H34 | −5.36 | −1.94 | 3.42 | 2.33 |
| H36 | −5.40 | −1.93 | 3.47 | 2.35 |
| H37 | −5.43 | −1.97 | 3.46 | 2.35 |
| H41 | −5.34 | −1.98 | 3.36 | 2.32 |
| H43 | −5.37 | −1.89 | 3.48 | 2.35 |
| H71 | −5.38 | −1.92 | 3.46 | 2.34 |
| Comparative | −5.44 | −1.68 | 3.76 | 2.33 |
| compound 1 | | | | |

In Table 1, Eg=LUMO-HOMO.

As can be seen from Table 1, the compounds H2, H8, H15, H33, H37, H41 and H43 of the present disclosure, compared with the comparative compound 1, have deeper LUMO energy levels (<−1.70 eV), which helps the compounds of the present disclosure to match a material of an adjacent layer, and due to that the deeper the LUMO energy level, the easier electrons generated from a cathode are to be injected and transported, thus also helps to reduce the turn-on threshold and working voltage of an element and reduce power consumption of the element; have deeper HOMO energy levels (<−5.25) eV), which helps to block holes; and, have higher lowest triplet energy levels (>2.30 eV), which helps to block excitons, confine the excitons in a light-emitting layer, and improve light-emitting efficiency of the element.

The following are several examples of applications of the organic compounds of the present disclosure in OLED elements.

### Application Example 1

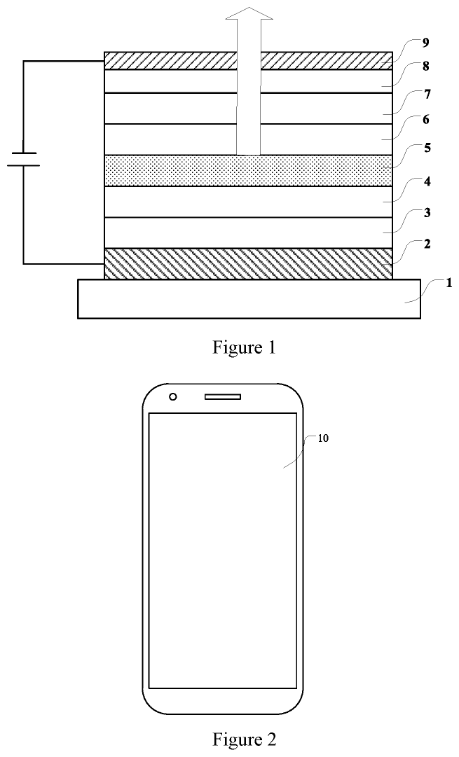

This application example provides an OLED element, whose structure is shown in FIG. 1. The OLED element includes a substrate 1, an anode 2, a hole injection layer 3, a first hole transport layer 4, a second hole transport layer 5, a light-emitting layer 6, a hole blocking layer 7, an electron transport layer 8 and a cathode 9 which are stacked in sequence. The arrow in FIG. 1 represents the direction in which the element emits light.

The OLED element was prepared by specific steps described below.

(1) A glass substrate with an indium tin oxide (ITO) anode (having a thickness of 15 nm) was cut to give a size of 50 mm×50 mm×0.7 mm, sonicated in isopropyl alcohol and deionized water for 30 minutes separately, and cleaned under ozone for 10 minutes. The cleaned glass substrate was installed onto a vacuum deposition apparatus.

(2) A hole injection layer material Compound b and a P-doping material Compound a were co-deposited with a doping ratio of 3% (a mass ratio) and a thickness of 5 nm by means of vacuum evaporation on the ITO anode 2, to serve as a hole injection layer 3.

(3) A hole transport material Compound b was deposited with a thickness of 100 nm by means of vacuum evaporation on the hole injection layer 3, to serve as a first hole transport layer 4.

(4) A hole transport material Compound d was deposited with a thickness of 5 nm by means of vacuum evaporation on the first hole transport layer 4, to serve as a second hole transport layer 5.

(5) A light-emitting host material Compound e and a doping material Compound f were co-deposited with a doping ratio of 3% (a mass ratio) and a thickness of 30 nm by means of vacuum evaporation on the second hole transport layer 5, to serve as a light-emitting layer 6.

(6) Compound g was deposited with a thickness of 30 nm by means of vacuum evaporation on the light-emitting layer 6, to serve as a hole blocking layer 7.

(7) Compound H2 and an N-doping material Compound h were co-deposited with a doping ratio of 1:1 and a thickness of 5 nm by means of vacuum evaporation on the hole blocking layer 7, to serve as an electron transport layer 8.

(8) A magnesium-silver electrode with a Mg:Ag mass ratio of 1:9 and a thickness of 10 nm was deposited by means of vacuum evaporation on the electron transport layer 8, to serve as a cathode 9.

The compounds used for preparing the OLED element were as follows:

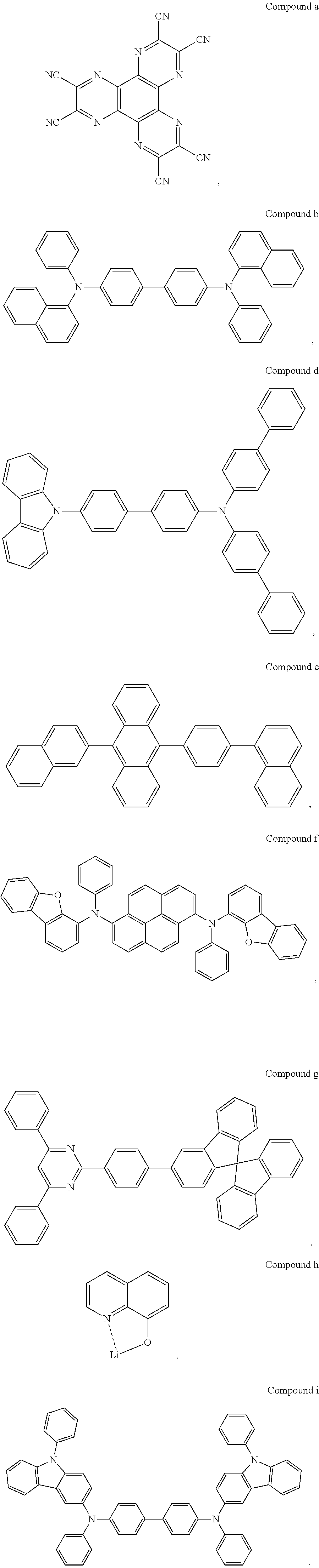

### Application Example 2

This application example provides an OLED element and differs from Application Example 1 in that Compound H2 in step (7) was replaced with Compound H8 on the premise that other preparation steps were the same.

### Application Example 3

This application example provides an OLED element and differs from Application Example 1 in that Compound H2 was replaced with Compound H15 on the premise that other preparation steps were the same.

### Application Example 4

This application example provides an OLED element and differs from Application Example 1 in that Compound H2 was replaced with Compound H31 on the premise that other preparation steps were the same.

### Application Example 5

This application example provides an OLED element and differs from Application Example 1 in that Compound H2 was replaced with Compound H33 on the premise that other preparation steps were the same.

### Application Example 6

This application example provides an OLED element and differs from Application Example 1 in that Compound H2 was replaced with Compound H34 on the premise that other preparation steps were the same.

### Application Example 7

This application example provides an OLED element and differs from Application Example 1 in that Compound H2 was replaced with Compound H36 on the premise that other preparation steps were the same.

### Application Example 8

This application example provides an OLED element and differs from Application Example 1 in that Compound H2 was replaced with Compound H37 on the premise that other preparation steps were the same.

### Application Example 9

This application example provides an OLED element and differs from Application Example 1 in that Compound H2 was replaced with Compound H41 on the premise that other preparation steps were the same.

### Application Example 10

This application example provides an OLED element and differs from Application Example 1 in that Compound H2 was replaced with Compound H43 on the premise that other preparation steps were the same.

### Application Example 11

This application example provides an OLED element and differs from Application Example 1 in that Compound H2 was replaced with Compound H71 on the premise that other preparation steps were the same.

### Comparative Example 1

This example provides an OLED element and differs from Application Example 1 in that Compound H2 was replaced with Comparative compound 1

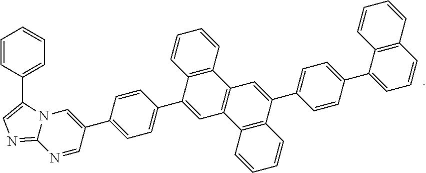 [SMILES: c1ccc(-c2cnc3ncc(-c4ccc(-c5cc6c7ccccc7c(-c7ccc(-c8cccc9ccccc89)cc7)cc6c6ccccc56)cc4)cn23)cc1]

Performance evaluation of OLED elements

A Keithley 2365A digital nanovoltmeter was used for testing currents of the OLED elements at different voltages, and then the currents were divided by a light-emitting area to obtain current densities of the OLED elements at different voltages. A Konicaminolta CS-2000 spectroradiometer was used for testing the brightness and radiant energy flux densities of the OLED elements at different voltages. According to the current densities and brightness of the OLED elements at different voltages, a working voltage and current efficiency (cd/A) at the same current density (10 mA/cm²) were obtained, where Von was the turn-on voltage when the brightness was 1 cd/m². Lifetime LT95 was obtained by measuring the time it took for the brightness of the OLED element to reach 95% of its initial brightness (under a testing condition of 500 mA/cm²). E/CIEy refers to a blue index in blue light and is also a parameter to measure blue light-emitting efficiency, where E refers to the current efficiency, and CIEy refers to an ordinate color point obtained by inputting the half-peak width of emission of the element into CIE1930 software. Test data is shown in Table 2.

TABLE 2

| | Material of | | | |
|---|---|---|---|---|
| | an Electron | | | Lifetime |
| OLED Element | Transport Layer | Vₒₙ (V) | E/CIEy | LT95 (h) |
| Application | Compound H2 | 3.87 | 150.1 | 67 |
| Example 1 | | | | |
| Application | Compound H8 | 3.85 | 152.2 | 66 |
| Example 2 | | | | |
| Application | Compound H15 | 3.84 | 151.8 | 68 |
| Example 3 | | | | |
| Application | Compound H31 | 3.81 | 152.0 | 64 |
| Example 4 | | | | |
| Application | Compound H33 | 3.83 | 150.9 | 67 |
| Example 5 | | | | |
| Application | Compound H34 | 3.84 | 152.7 | 65 |
| Example 6 | | | | |
| Application | Compound H36 | 3.85 | 151.3 | 66 |
| Example 7 | | | | |
| Application | Compound H37 | 3.83 | 152.4 | 68 |
| Example 8 | | | | |
| Application | Compound H41 | 3.85 | 151.5 | 65 |
| Example 9 | | | | |
| Application | Compound H43 | 3.82 | 153.0 | 66 |
| Example 10 | | | | |
| Application | Compound H71 | 3.84 | 152.9 | 67 |
| Example 11 | | | | |
| Comparative | Comparative | 3.98 | 141.8 | 60 |
| Example 1 | compound 1 | | | |

As can be seen from Table 2, compared with Comparative Example 1, Application Examples 1 to 7 have lower working voltages, higher blue indexes (light-emitting efficiency), and longer lifetimes, which are increased by about 3.8%, 7.2% and 10%, respectively. This is mainly because the compounds of the present disclosure have deeper LUMO energy levels with smaller band gap differences between LUMO energy levels of materials in adjacent layers, which is advantageous for the effective injection and transport of electrons. Meanwhile, the improvement of the element lifetimes is also based on the fact that the compounds of the present disclosure have deeper LUMO energy levels and can be better complexed with an N-dopant.

Another application example of the present disclosure provides a display panel including the OLED element described above.

Another application example of the present disclosure provides an organic light-emitting display device including the display panel described above.

In the present disclosure, the OLED element may be applied in the organic light-emitting display device. The organic light-emitting display device may be a display of a mobile phone, computer, television, smart watch, smart car, and VR or AR helmet, and displays of various smart apparatuses, etc. FIG. 2 is a schematic diagram of an organic light-emitting display device according to an application example of the present disclosure, where 10 is the display of the mobile phone.

What is claimed is:

1. A compound, having a structure represented by Formula I: 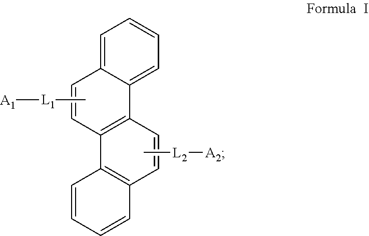
wherein L₁ and L₂ are each independently selected from any one of a single bond, substituted or unsubstituted C6 to C40 aryl or substituted or unsubstituted C3 to C40 heteroaryl;
A₁ and A₂ are each independently selected from any one of substituted or unsubstituted C6 to C40 aryl or substituted or unsubstituted C3 to C40 heteroaryl, and at least one of A₁ and A₂ is selected from any one of group 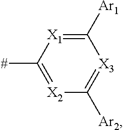
wherein # represents a position where the group is joined;
wherein X₁, X₂ and X₃ are each independently a N atom or —CH, and at least one of X₁, X₂ and X₃ is a N atom;
Ar₁ and Ar₂ are each independently selected from any one of substituted or unsubstituted C6 to C40 aryl or substituted or unsubstituted C3 to C40 heteroaryl; and
when a substituent is present in the above groups, the substituent is methyl, ethyl, isopropyl, t-butyl, methoxy, cyano, phenyl, biphenyl, terphenyl, naphthyl, anthryl, phenanthryl, pyridyl, pyrimidyl, triazinyl, carbazolyl, dibenzofuryl or dibenzothienyl.

2. The compound according to claim 1, having a structure represented by Formula II:

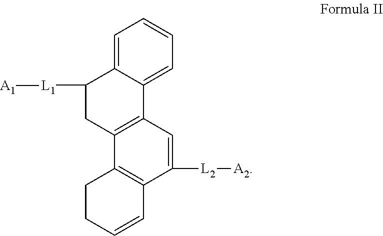

3. The compound according to claim 1, wherein L₁ and L₂ are each independently selected from any one of a single bond, phenylene, biphenylene, naphthylene, anthrylene, furylene, thienylene, pyridylene, pyrimidylene, triazinylene or fluorenylene.

4. The compound according to claim 1, wherein A₁ and A₂ are each independently selected from any one of phenyl, naphthyl, anthryl, pyridyl, pyrimidyl, triazinyl, dibenzofuryl, dibenzothienyl, carbazolyl, fluorenyl, spirofluorenyl or 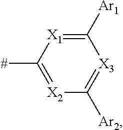
and at least one of A₁ and A₂ is selected from any one of

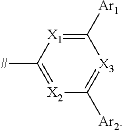

5. The compound according to claim 1, wherein Ar₁ and Ar₂ are each independently selected from any one of phenyl, biphenyl, terphenyl, naphthyl, anthryl, phenanthryl, pyridyl, pyrimidyl, triazinyl, dibenzofuryl, dibenzothienyl, carbazolyl, fluorenyl or spirofluorenyl.

6. The compound according to claim 1, wherein the group 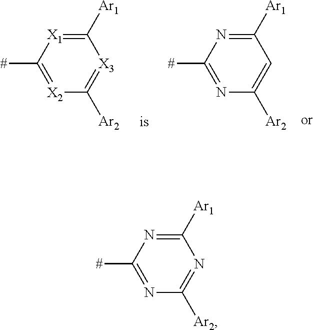
wherein # represents the position where the group is joined.

7. The compound according to claim 2, wherein L₁ and L₂ are each independently selected from any one of a single bond, phenylene, biphenylene, naphthylene, anthrylene, furylene, thienylene, pyridylene, pyrimidylene, triazinylene or fluorenylene.

8. The compound according to claim 2, wherein A₁ and A₂ are each independently selected from any one of phenyl, naphthyl, anthryl, pyridyl, pyrimidyl, triazinyl, dibenzofuryl, dibenzothienyl, carbazolyl, fluorenyl, spirofluorenyl or 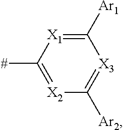
and at least one of A₁ and A₂ is selected from any one of

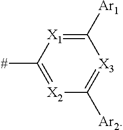

9. The compound according to claim 2, wherein Ar₁ and Ar₂ are each independently selected from any one of phenyl, biphenyl, terphenyl, naphthyl, anthryl, phenanthryl, pyridyl, pyrimidyl, triazinyl, dibenzofuryl, dibenzothienyl, carbazolyl, fluorenyl or spirofluorenyl.

10. The compound according to claim 2, wherein the group 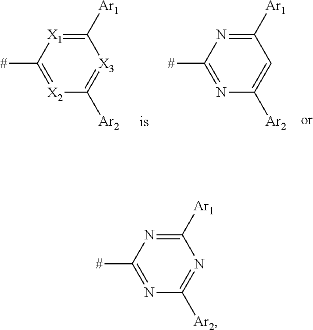
wherein # represents the position where the group is joined.

11. The compound according to claim 2, having a structure represented by Formula III or Formula IV: 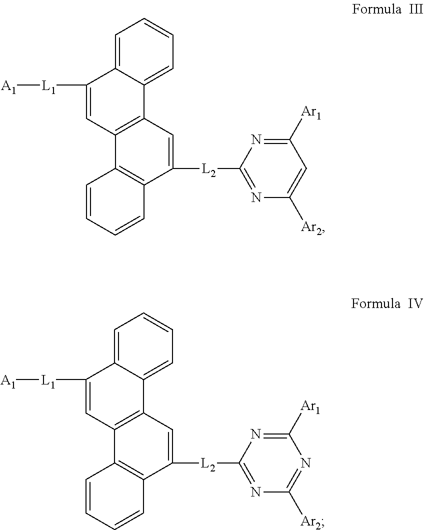
wherein L₁ and L₂ are each independently selected from any one of a single bond, phenylene, biphenylene, naphthylene, anthrylene, furylene, thienylene, pyridylene, pyrimidylene, triazinylene or fluorenylene;
A₁ is selected from any one of phenyl, naphthyl, anthryl, pyridyl, pyrimidyl, triazinyl, dibenzofuryl, dibenzothienyl, carbazolyl, fluorenyl, spirofluorenyl, 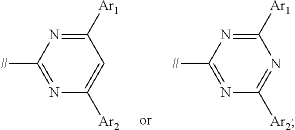
and
Ar₁ and Ar₂ are each independently selected from any one of phenyl, biphenyl, terphenyl, naphthyl, anthryl, phenanthryl, pyridyl, pyrimidyl, triazinyl, dibenzofuryl, dibenzothienyl, carbazolyl, fluorenyl or spirofluorenyl.

12. The compound according to claim 11, having a structure represented by Formula IV;
wherein L₁ and L₂ are each independently a single bond, phenylene or naphthylene;
A₁ is selected from any one of phenyl, naphthyl, pyridyl, dibenzofuryl, dibenzothienyl, carbazolyl, fluorenyl or 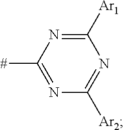
Ar₁ and Ar₂ are each independently selected from any one of phenyl, biphenyl, pyridyl, dibenzofuryl, dibenzothienyl, carbazolyl or fluorenyl.

13. The compound according to claim 1, wherein the compound is selected from any one of following compounds H1 to H81:

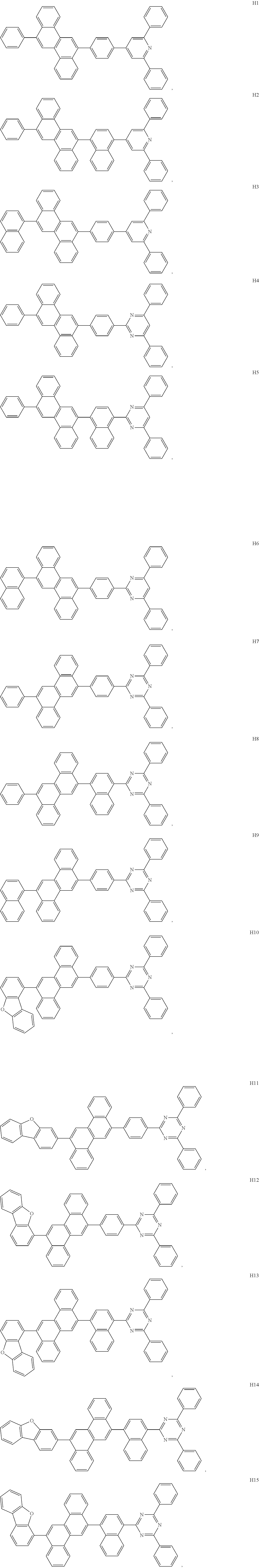

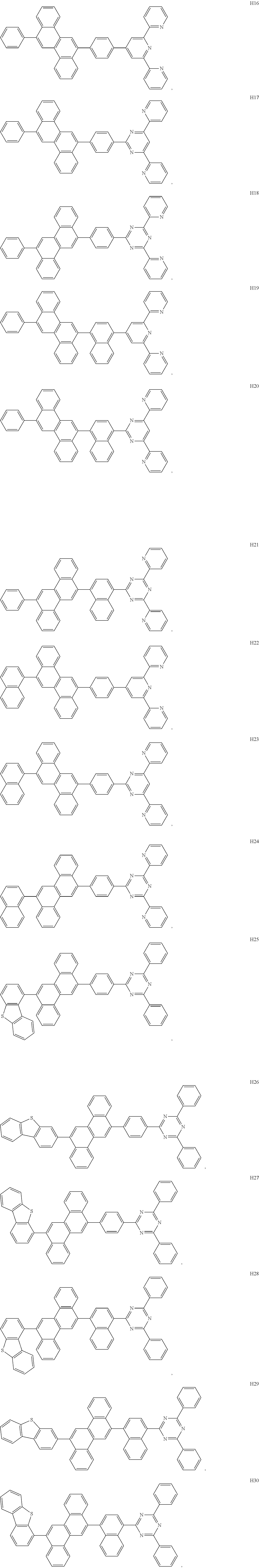

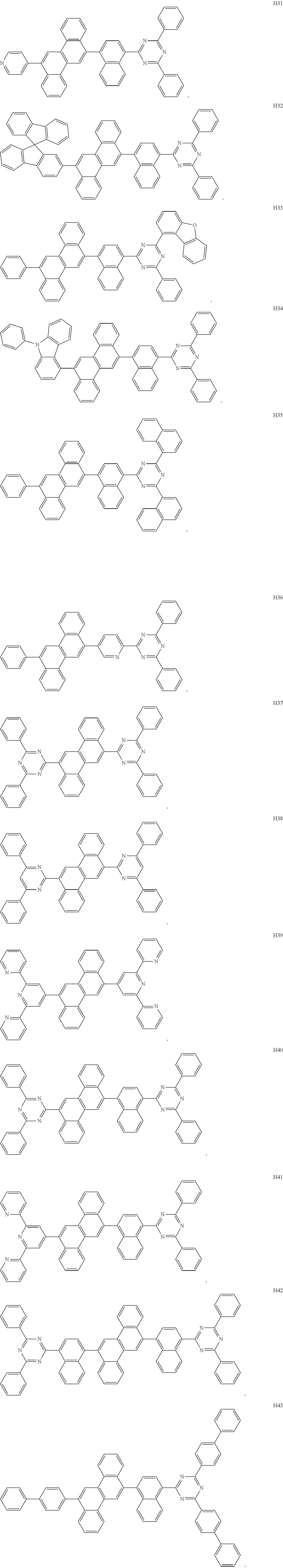

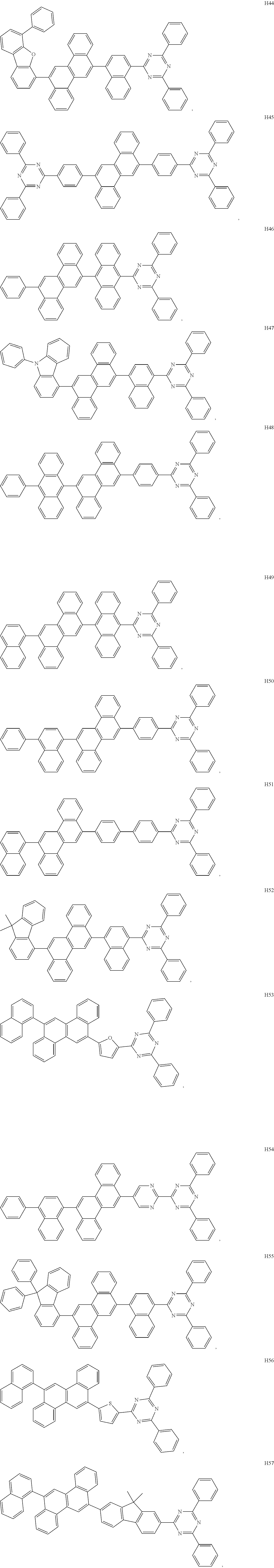

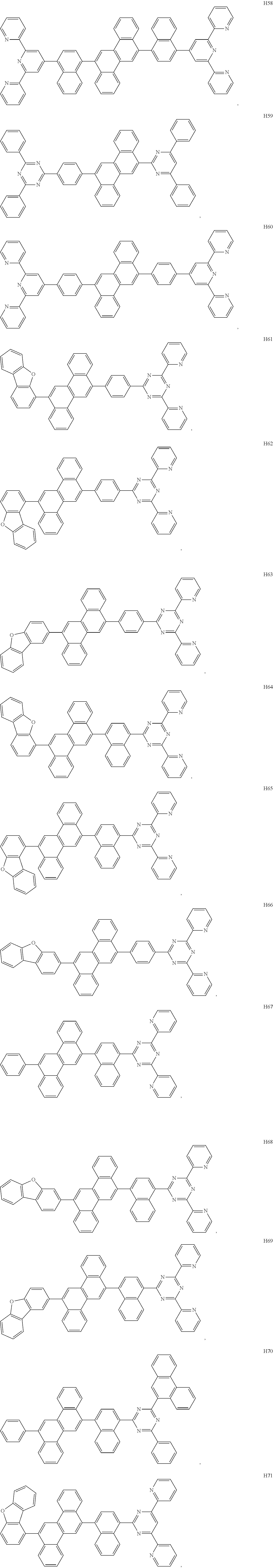

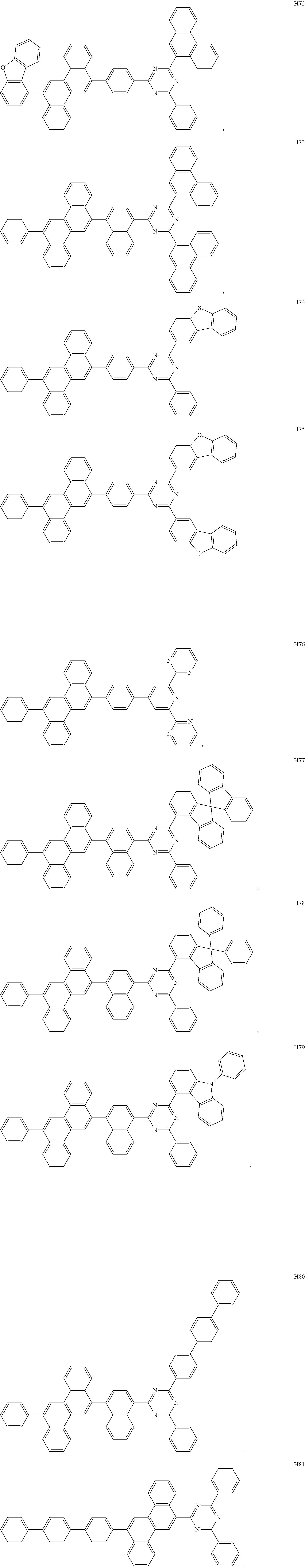

14. An organic light-emitting element, comprising an anode, a cathode and an organic thin film layer disposed between the anode and the cathode; wherein the organic thin film layer comprises any one or a combination of at least two of a light-emitting layer, an electron transport layer and a hole blocking layer, and comprises at least a light-emitting layer;
wherein at least one of the light-emitting layer, the electron transport layer or the hole blocking layer contains at least one of the compounds each according to claim 1, wherein the compound having a structure of Formula I.

15. The organic light-emitting element according to claim 14, wherein the organic thin film layer further comprises any one or a combination of at least two of a hole injection layer, a hole transport layer, an electron blocking layer and an electron injection layer.

16. The organic light-emitting element according to claim 14, wherein the light-emitting layer comprises a host material and a light-emitting material, wherein the host material of the light-emitting layer comprises any one or a combination of at least two of the compounds each having a structure of Formula I.

17. The organic light-emitting element according to claim 14, wherein the electron transport layer comprises a material selected from any one or a combination of at least two of the compounds each having a structure of Formula I; or
the electron transport layer comprises a host material and a guest material, wherein the host material of the electron transport layer is selected from any one or a combination of at least two of the compounds each having a structure of Formula I.

18. The organic light-emitting element according to claim 10, wherein the hole blocking layer comprises a material selected from any one or a combination of at least two of the compounds each having a structure of Formula I, and the light-emitting layer comprises a light-emitting material having a lowest triplet energy level lower than the lowest triplet energy level of the compound.

19. A display panel, comprising the organic light-emitting element according to claim 14, wherein the display panel is optionally used in a display device.
